(19) United States
(12) Patent Application Publication
(10) Pub. No.: US 2008/0114055 A1
(43) Pub. Date: May 15, 2008

(54) Thioxanthone Compounds to Reverse Weight Loss

(75) Inventor: Zoltan Kiss, Austin, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: ZOLTAN LABORATORIES LLC, Austin, MN (US)

(21) Appl. No.: 11/558,533

(22) Filed: Nov. 10, 2006

(51) Int. Cl. A61K 31/382; A61P 35/00; A61P 31/00; A61P 11/00

(52) U.S. Cl. 514/437

(57) ABSTRACT

One embodiment of the invention uses thioxanthone compounds containing a choline or choline analogue moiety, such as CCcompound1 or [3-(3,4-Dimethyl-9-oxo-9H-thioxanthen-2-yloxy)-2-hydroxypropyl]trimethyl-ammonium chloride, to reduce or reverse body weight loss in various mouse models of human tumors. Another embodiment of the invention discloses a method for reducing or reversing cachexia or disease-induced body weight loss in human and other mammals using thioxanthone compounds containing a choline analogue moiety.

### FIELD OF INVENTION

Use of thioxanthone compounds containing a choline or choline analogue moiety to reverse loss of body weight in various mouse models of human tumors and by implication in human subjects as well as any other subjects who experience unintended weight loss.

### BACKGROUND

Cachexia is a complex metabolic disorder resulting in progressive loss of weight. Such unintended weight loss occurs in about 80% of subjects with advanced cancer and is responsible for the death of about 20% of cancer subjects. Weight loss also decreases the chance to respond to chemotherapy and renders the cachectic subjects more prone to toxic side effects. Cachexia is also associated with other diseases such as AIDS, congestive heart failure, chronic obstructive pulmonary disease, cystic fibrosis, Crohn's disease, infections, rheumatoid arthritis, tuberculosis, and it frequently burdens the elderly. Cachexia affects the metabolism in both skeletal tissue and adipose tissue. Loss of body weight is mainly due to increased lipolysis and decreased lipogenesis in the adipose tissue as well as reduced protein synthesis and increased protein degradation in the skeletal muscle [Gordon, J. N., Green, S. R. and Goggin, P. M. (2005) “Cancer cachexia,” Q. J. Med. 98, 779-788].

Presently there is an uncertainty over what causes cachexia. The etiology of cachexia is multifactorial; however, there is no consensus on the nature and relative importance of cachexia-inducing factors. In addition, there are yet to be discovered additional factors that contribute to this phenomenon. Finally, it is also not clear what kind of mechanisms exist in the healthy tissues to counter the tumor-derived cachexia-inducing signals.

### SUMMARY OF THE INVENTION

One embodiment of the invention is directed to a pharmaceutically acceptable composition to reduce or reverse cachexia or disease-induced body weight loss comprising a therapeutically effective amount of a thioxanthone compound containing a choline or choline analogue moiety. In some embodiments of the invention, the thioxanthone compound is [3-(3,4-Dimethyl-9-oxo-9H-thioxanthen-2-yloxy)-2-hydroxypropyl]trimethyl-ammonium chloride. Another embodiment of the invention is directed toward a method for reducing or reversing cachexia or disease-induced body weight loss comprising, administering to a subject a pharmaceutically acceptable composition comprising a therapeutically effective amount of a thioxanthone compound containing a choline or choline analogue moiety.

### DETAILED DESCRIPTION

An anti-cachectic effect of CCcompound1, a trivial name for [3-(3,4-Dimethyl-9-oxo-9H-thioxanthen-2-yloxy)-2-hydroxypropyl]trimethyl-ammonium chloride, was demonstrated in four known human xenograft models including estrogen receptor positive T47D human breast cancer [Devarajan, E., Sahin, A. A., Chen, J. S., Krishnamurthy, R. R., Aggarwal, N., Brun, A. M., Sapino, A., Zhang, F., Sharma, D., Yang, X. H., Tora, A. D. and Mehta, K. (2002), “Down-regulation of caspase 3 in breast cancer: a possible mechanism for chemoresistance,” Oncogene, 21, 8843-8851], PC3 human prostate cancer [Srivastava, S. K., Xiao, D., Lew, K. L., Hershberger, P., Kokkinakis, D. M., Johnson, C. S., Trump, D. L. and Singh, S. V. (2003), “Allyl isothiocyanate, a constituent of cruciferous vegetables, inhibits growth of PC-3 human prostate cancer xenografts in vivo,” Carcinogenesis, 24, 1665-1670], HL60 human leukemia [Cappellini, A., Tabellini, G., Zweyer, M., Bortul, R., Tazzari, P. L., Billi, A. M., Fala, F., Cocco, L. and Martelli, A. M. (2003), “The phosphoinositide 3-kinase/Akt pathway regulates cell cycle progression of HL60 human leukemia cells through cytoplasmic relocalization of the cyclin-dependent kinase inhibitor p27^{Kip1} and control cyclin D1 expression,” Leukemia, 17, 2157-2167] and HT-168 human melanoma [Szincsak, N., Hegyesi, H., Hunyadi, J., Falus, A. and Juhasz, I. (2002), “Different H2 receptor antihistamines dissimilarly retard the growth of xenografted human melanoma cells in immunodeficient mice,” Cell Biol. Internat., 26, 833-836] tumors, all developed in a homozygous line of C.B.-171cr scid/scid (seriously compromised immunodeficient) mice.

All the above tumors induced significant reductions in body weight. “Body weight” is defined as the total weight of a subject minus the tumor weight. In each case, repeated subcutaneous injections of CCcompound1 resulted in the reversal of body weight loss. A “subject” includes a human or any other mammal.

In one embodiment, the invention uses a treatment regimen for the reduction or reversal of body weight loss in a tumor bearing mammal, particularly in a human, that includes periodically administering a therapeutically effective amount of thioxanthone compound containing a choline or choline analogue moiety.

In another embodiment, the treatment regimen involves regular oral administration of a thioxanthone compound in the form of a tablet or capsule, or in any other form suitable for oral intake.

In yet another embodiment, the treatment regimen involves regular injections of a suspended thioxanthone compound via one of the applicable injection routes including controlled release via an implanted minipump.

In case of cancer, the treatments with a thioxanthone compound via any of the application routes may occur prior to or after other therapies, or may be applied as part of a more complex therapy, for example chemotherapy or radiation therapy. In some embodiments, use of a thioxanthone compound occurs during the resting period between two cycles of treatment.

Other embodiments of the invention also use a thioxanthone compound to reduce or prevent unintended weight loss in other diseases such as AIDS, congestive heart failure, chronic obstructive pulmonary disease, cystic fibrosis, Crohn's disease, infections, rheumatoid arthritis, tuberculosis and others as well in elderly persons who develop cachexia without obvious disease or starvation.

Active Compounds

Embodiments of the present invention use the chemically synthesized compound, [3-(3,4-dimethyl-9-oxo-9H-thioxanthen-2-yloxy)-2-hydroxypropyl]trimethyl-ammonium chloride or CCcompound1, the structure of which is shown below, or related compounds to reduce or prevent tumor induced body weight loss.

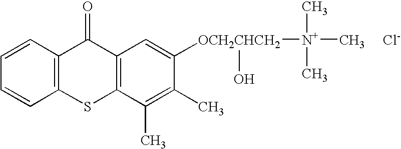

CCcompound1 can be purchased from Sigma-Aldrich in 2001 (catalog number: 40,633-3 in the 2001 Aldrich catalog).

CCcompound1 can be cleaved by an appropriate enzyme (an ether hydrolyse) to yield choline, which is a precursor for phosphatidylcholine (PC) synthesis. The growing tumor tissue develops a high capacity to transport choline into tumor cells that deprives the normal tissues of this important PC precursor. CCcompound1 may help survive normal tissues by providing choline for PC synthesis.

Ethanolamine, monomethylethanolamine and dimethylethanolamine as well as various trialkyl analogues of choline may substitute for choline in the thioxanthone molecule because the corresponding phospholipids either can be methylated to form PC or the resulting phospholipid analogues are biologically equivalent or near equivalent with PC.

The thioxanthone-based structural analogues of CCcompound1 that are likely to also exert anti-cachectic effects as well as their synthesis were described in filed U.S. patent application Ser. No. 11/458,502, filed on Jul. 19, 2006, entitled “Compounds and compositions to control abnormal cell growth,” incorporated herein by reference. See also U.S. application Ser. No. 11,465,876, filed Aug. 21, 2006, entitled “Compositions to Reduce or Prevent Skin Cancer,” incorporated herein by reference. They contain one choline or a choline analogue moiety group attached to the 2nd or 9th position in the thioxanthone moiety. The thioxanthone compounds that may mimic the anti-cachectic effect of CCcompound1 are also listed in TABLE 1.

TABLE 1
CCompound1 and a representative List of Related Thioxanthone
Compounds Capable of Exerting Anti-Cachectic Effects.

| Trivial name | Chemical name | Structure |
|---|---|---|
| CCcompound1 | [3-(3,4-Dimethyl-9-oxo-9H-thioxanthen-2-yloxy)-2-hydroxypropyl]trimethyl-ammonium chloride | 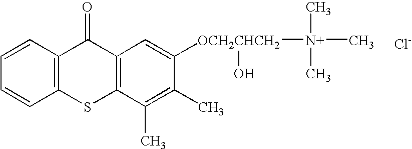 |
| CCcompound2 | N,N,N-Trimethyl-2-[(9-oxo-9H-thioxanthen-2-yl)methoxy]-ethanaminiumiodide | 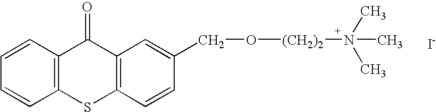 |
| CCcompound3 | N,N-Diethyl-N-methyl-2-[9-oxo-9H-CCDTHTthioxanthen-2-yl)methoxy]-ethanaminium iodide | 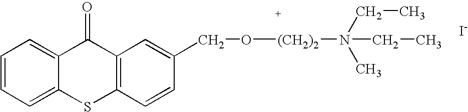 |
| CCcompound4 | N,N,N-Triethyl-2-[(9-oxo-9H-thioxanthen-2-yl)methoxy]-ethanaminiumiodide | 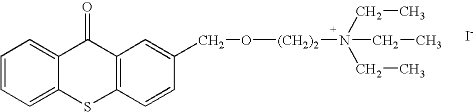 |
| CCcompound5 | N-Ethyl-N,N-dimethyl-2-[(9-oxo-9H-thioxanthen-2-yl)methoxy]-ethanaminiumiodide | 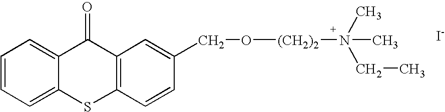 |
| CCcompound6 | 2-{[2-(Diethylamino)ethoxy]methyl}-9H-thioxanthen-9-onehydrochloride | 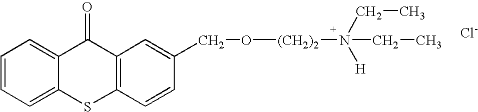 |
| CCcompound7 | N,N,N-Trimethyl-2-[(9-oxo-9H-thioxanthen-2-yl)methoxy]-propan-1-aminium iodide | 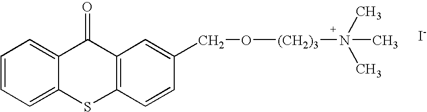 |
| CCcompound8 | 2-{[2-(Methylamino)propoxy]methyl}-9H-thioxanthen-9-onehydrochloride | 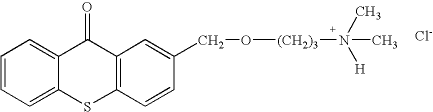 |
| CCcompound9 | N,N,N-Triethyl-3-[(9-oxo-9H-thioxanthen-2-yl)methoxy]-propan-1-aminium iodide | 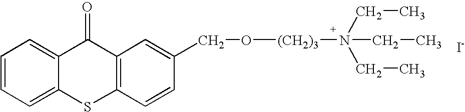 |
| CCcompound10 | N,N-Diethyl-N-methyl-3-[(9-oxo-9H-thioxanthen-2-yl)methoxy]-propan-1-aminium iodide | 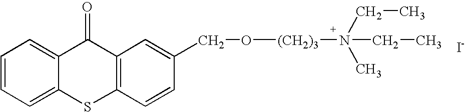 |
| CCcompound11 | N,N-Dimethyl-N-ethyl-3-[(9-oxo-9H-thioxanthen-2-yl)methoxy]-propan-1-aminium iodide | 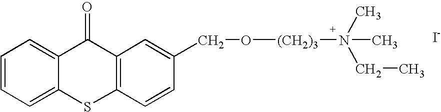 |
| CCcompound12 | 2-{[3-(Diethylamino)propoxy]methyl}-9H-thioxanthen-9-onehydrochloride | 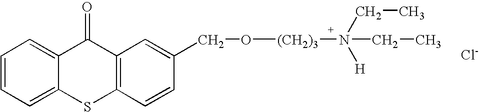 |
| CCcompound13 | 2-Hydroxy-N,N-dimethyl-N-[(9-oxo-9H-thioxanthen-2-yl)methyl]-ethanaminiumbromide | 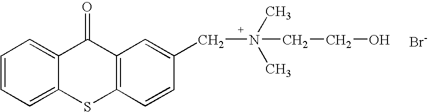 |
| CCcompound14 | 2-Hydroxy-N,N-Diethyl-N-[(9-oxo-9H-thioxanthen-2-yl)methyl]-ethanaminiumbromide | 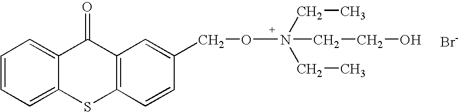 |
| CCcompound15 | 3-Hydroxy-N,N-dimethyl-N-[(9-oxo-9H-thioxanthen-2-yl)methyl]propan-1-aminium bromide | 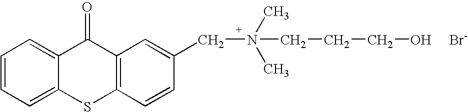 |
| CCcompound16 | 3-Hydroxy-N,N-diethyl-N-[(9-oxo-9H-thioxanthen-2-yl)methyl]-propan-1-aminium bromide | 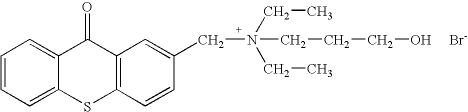 |
| CCcompound17 | 3-(9-hydroxy-9H-thioxanthen-9-yl)-N,N,N-trimethyl-propan-1-aminiumiodide | 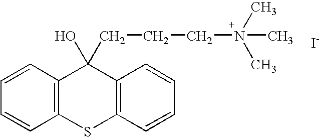 |
| CCcompound18 | 3-(9-hydroxy-9H-selenoxanthen-9-yl)-N,N,N-trimethyl-propan-1-aminiumiodide | 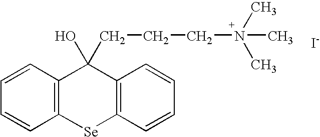 |
| CCcompound19 | N,N,N-trimethyl-3-(9H-thioxanthen-9-ylidene)-propan-1-aminium iodide | 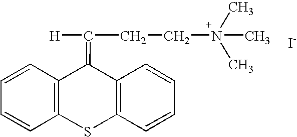 |
| CCcompound20 | N,N,N-trimethyl-3-(9H-selenoxanthen-9-ylidene)-propan-1-aminium iodide | 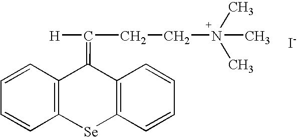 |
| CCcompound21 | N,N,N-trimethyl-3-(2-methyl-9H-thioxanthen-9-ylidene)-propan-1-aminium iodide | 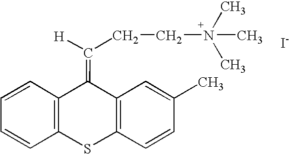 |
| CCcompound22 | N,N-Dimethyl-N-ethyl-3-(2-methyl-9H-thioxanthen-9-ylidene)-propan-1-aminiumiodide | 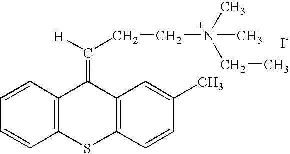 |
| CCcompound23 | N,N-Dimethyl-N-ethyl-3-(2-methyl-9H-thioxanthen-9-ylidene)-propan-1-aminiumiodide | 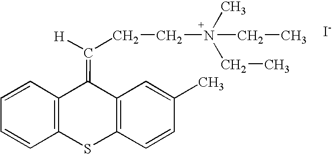 |
| CCcompound24 | N,N-Dimethyl-N-allyl-3-(2-methyl-9H-thioxanthen-9-ylidene)-propan-1-aminiumbromide | 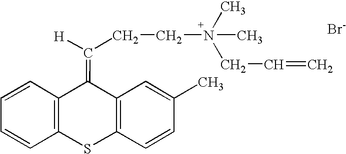 |
| CCcompound25 | N,N,N-trimethyl-3-(2-methyl-9H-thioxanthen-9-ylidene)-propan-1-aminiumiodide | 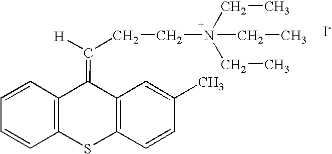 |
| CCcompound26 | N,N-Dimethyl-N-allyl-3-(2-methyl-9H-thioxanthen-9-ylidene)-propan-1-aminiumbromide | 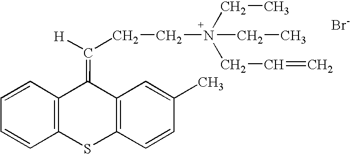 |

Methods of Use

Since CCcompound1 is fully water-soluble at the concentration used in vivo, oral and rectal applications to subjects who experience unintended weight loss are methods of delivering it in a therapeutically effective dose. “Therapeutically effective” means a dose that causes a measurable increase in body weight in a subject who experiences weight loss.

While only CCcompound1 is discussed in the application, it should be understood that CCcompound1 may be replaced with any of its related thioxanthone compound such as, for example, those compounds that are listed in Table 1.

In one embodiment of the invention, CCcompound1 is in the form of a tablet, shaped gel-like composition (like a capsule), a liquid, or the like. In each case, CCcompound1 is mixed with one or more carriers chosen by one having ordinary skill in the art to best suit the goal of either oral or rectal treatment. In addition to CCcompound1, the tablet or gel may contain any component that is presently used in the pharmaceutical field to ensure firmness, stability, solubility and appropriate taste. Any additional component of the tablet or gel will be chemically inert; i.e., it will not participate in a chemical reaction with the CCcompound1 or the other additives.

In addition to the active component and tablet-forming components, the tablet or gel may contain other biologically active compounds such as, for example, an anti-cachectic agent(s) or agents that promote(s) the reversing actions of the CCcompound1 on body weight loss. The list of these agents may include inhibitors of the ubiquitin-proteasome pathway (accounting for most protein degradation in the muscle) such as lactacystin [Lazarus, D. D., Destree, A. T., Mazzola, L. M., McCormack, T. A., Dick, L. R., Xu, B., Huang, J. Q., Pierce, J. W., Read, M. A., Coggins, M. B., Solomon, V., Goldberg, A. L., Brand, S. J. and Elliott, P. J. (1999), “A new model of cancer cachexia: contribution of the ubiquitin-proteasome pathway,” Am. J. Physiol., 277, E332-E341], β-hydroxy-β-methylbutyrate [Smith, H. J., Mukeiji, P. and Tisdale, M. J. (2005), “Attenuation of proteasome-induced proteolysis in skeletal muscle by β-hydroxy-β-methylbutyrate in cancer-induced muscle loss,” Cancer Res., 65, 277-283], and eicosapentaenoic acid [Whitehouse, A. S., Smith, H. J., Drake, J. L. and Tisdale, M. J. (2001), “Mechanism of attenuation of skeletal muscle protein catabolism in cancer cachexia by eicosapentaenoic acid,” Cancer Res., 61, 3604-3609] as well as the cyclooxygenase-2 inhibitor celecoxib [Davis, T. W., Zweifel, B. S., O'Neal, J. M., Heuvelman, D. M., Abegg, A. L., Hendrich, T. O. and Masferrer, J. L. (2004), “Inhibition of cyclooxygenase-2 by celecoxib reverses tumor-induced wasting,” J. Pharmacol. Exp. Therap., 308, 929-934] and an appetite stimulator such as megesterol acetate.

In case of either oral or rectal application, one dose may contain about 20-1,000-mg CCcompound1 per m² body surface. In a suitable embodiment, one dose for oral or rectal application contains about 50-500-mg CCcompound1 m² body surface.

In another embodiment, CCcompound1 is suspended in a liquid biocompatible carrier, such as physiological saline (0.9% NaCl), and injected via one of the systemic routes such as, for example, intravenous, intraarterial, intraperitoneal, subcutaneous, intramuscular, or intradermal. CCcompound1 may also be administered via infusion or an implanted minipump over a prolonged period of time in a controlled fashion.

In case of applications by injection, infusion, or minipump, one dose may contain between about 20-1,000-mg CCcompound per m² body surface. In a suitable embodiment, one dose for application by injection, infusion, or minipump contains about 50-500-mg CCcompound1 per m² body surface.

CCcompound1 can be administered via one or more of the above applicable routes for a suitable time period. The length of a suitable time period, which may be 1 week, 1 month, or as many months as necessary, is individually determined by the healthcare provider and depends on the severity of the situation and other factors. The healthcare provider also determines the frequency of the treatments. In some embodiments, CCcompound1 may be delivered three-times every 24 hours; in other cases, once per day or three-times a week. Each time, CCcompound1 may be delivered together with other anti-cachectic agents including, for example, those that were listed above.

Series of chemotherapy or radiotherapy given to the subject are scheduled so that in most cases there is a significant time period (usually 2-3 weeks) between two series of treatments. This so-called recovery period between the treatment series is established to allow the subject to recover, at least partially, from the usually serious side effects caused by the given treatment. Unfortunately, many subjects keep losing weight during the recovery period, which makes them even more vulnerable to the subsequent treatment. In some embodiments, CCcompound1 is administered during the recovery period, enabling the subject to better tolerate the next round of therapy. In other embodiments, CCcompound1 may be used at any time during any type of disease treatment. In yet other embodiments, the CCcompound1 may be used continuously with doses and frequencies as determined by the healthcare provider.

Accelerated loss of skeletal muscle and adipose tissue independent of nutrient intake is also a significant component of weight loss triggered by many chronic or end-stage diseases such as AIDS, congestive heart failure, chronic obstructive pulmonary disease, cystic fibrosis, Crohn's disease, infections, rheumatoid arthritis, tuberculosis and others. Cachexia also develops in a significant proportion of elderly persons without disease or starvation. Importantly, in all these cases a common mechanism to execute cachexia is increased degradation of muscle proteins via the adenosine triphosphate-dependent ubiquitin-proteasome proteolytic pathway [Kotler, D. P. (2000), “Cachexia,” Ann. Intern. Med. 133, 622-634]. Because CCcompound1 prevents protein degradation in cancer models to maintain body weight, and protein degradation is a common element of cachexia in all these diseases, CCcompound1 may reduce or reverse unintended body weight loss in all the above cited disease states.

### EXAMPLES

### Example 1

### Development and Treatment of Tumor Models

Tumors were developed in a homozygous line of C.B.-171 cr scid/scid adult female mice kept at specified pathogen free (SPF) hygienic levels. Suspensions of HL-60 human leukemia cells (about 1,500,000 cells), or approximately 0.1 cm³ volume of tumor tissue derived from HT-168 human melanoma, PC-3 human prostate adenocarcinoma, or T47D human breast carcinoma were implanted subcutaneously to develop the tumors. After 12-18 days when the tumor-bearing mice were first treated, the size of the tumors was in the 0.2-0.4 cm³ range. CCcompound1 was dissolved in physiological (0.9%) saline (NaCl) and applied subcutaneously in 50 μl volume. Tumor volume was determined by calipers in three dimensions; this technique is well known to one having ordinary skill in the art. Tumor volume was calculated according to the generally accepted formula: V=a²×b×π/6, where “a” and “b” mean the shortest and longest diameter, respectively, of the measured tumor. Tumor weight was calculated from tumor volume using a 0.7 (tumor weight) : 1 (tumor volume) ratio. Total weight and tumor volume/weight was measured regularly (at least every second day). In all tumor experiments, each treatment group consisted of five animals. Accordingly, all data are the average of five independent determinations (one determination for each animal).

### Example 2

### Effects of CCcompound1 on Body Weight as Well as Total and Tumor Weight in the HL60 Human Leukemia Tumor Model

HL60 human tumors were developed in mice as described above. Treatments with CCcompound1 were started on day 18 after tumor implantation and conducted for the first five days and then for three days with two treatment-free days inserted between the two series of treatments. The results, shown in TABLE 2, indicate that between day 18 and 27 after tumor implantation, the leukemia tumor caused about 4-g loss in body weight. While administration of CCcompound1 in the amount of 0.5-mg/kg reduced body weight loss, animals treated with 1-mg/kg or 2-mg/kg CCcompound1 actually gained weight. The results also show that higher amounts of CCcompound1 also reduced tumor weight about 45%. Overall the results indicate that in the HL60 human leukemia tumor model, CCcompound1 is a potent anti-cachectic agent, and its effects on body weight are accompanied by a significant anti-tumor effect.

TABLE 2
CCcompound1 prevents body weight loss in the HL60 tumor model.

Days after
tumor
transplantation

| CCcompound1 mg/kg | Weight (g) | 18 | 27 |
|---|---|---|---|
| 0 | Total weight | 26.3 | 25.2 |
| | Tumor weight | 0.6 | 3.4 |
| | Body weight | 25.7 | 21.8 |
| 0.5 | Total weight | 26.2 | 26.8 |
| | Tumor weight | 0.7 | 2.8 |
| | Body weight | 25.5 | 24.0 |
| 1.0 | Total weight | 26.2 | 30.3 |
| | Tumor weight | 0.4 | 1.9 |
| | Body weight | 25.8 | 28.4 |
| 2.0 | Total weight | 27.0 | 30.6 |
| | Tumor weight | 0.5 | 1.9 |
| | Body weight | 26.5 | 28.7 |

### Example 3

### Effects of CCcompound1 on Body Weight as Well as Total and Tumor Weight in the HT-168 Human Melanoma Model

HT-168 human tumors were developed in mice as described above. Treatments with CCcompound1 were started on day 17 after tumor implantation and conducted for the first five days and then for another five days with two treatment-free days inserted between the two series of treatments. The results, shown in TABLE 3, indicate that between day 17 and 30 after tumor implantation, the melanoma tumor caused a 2.7-g loss in body weight. Administration of CCcompound1 in the amount of 1-mg/kg was sufficient to reverse body weight loss, while animals that received 4-mg/kg of CCcompound1 gained almost 5-g. Treatment with 4-mg/kg of CCcompound1 also reduced tumor weight more than 50%. Overall the results indicate that in the HT-168 human melanoma model, CCcompound1 is an effective anti-cachectic agent while also exerting significant anti-tumor effects.

TABLE 3
CCcompound1 prevents body weight loss in the HT-168 tumor model.

Days after
tumor
transplantation

| CCcompound1 mg/kg | Weight (g) | 17 | 30 |
|---|---|---|---|
| 0 | Total weight | 24.0 | 23.6 |
| | Tumor weight | 0.2 | 2.5 |
| | Body weight | 23.8 | 21.1 |
| 1.0 | Total weight | 24.0 | 27.6 |
| | Tumor weight | 0.2 | 2.1 |
| | Body weight | 23.8 | 25.5 |
| 2.0 | Total weight | 23.1 | 25.8 |
| | Tumor weight | 0.2 | 1.8 |
| | Body weight | 22.9 | 24.0 |
| 4.0 | Total weight | 23.8 | 29.6 |
| | Tumor weight | 0.2 | 1.2 |
| | Body weight | 23.6 | 28.4 |

### Example 4

### Effects of CCcompound1 on Body Weight as Well as Total and Tumor Weight in the PC-3 Human Prostate Cancer Model

PC-3 human tumors were developed in mice as described above. Treatments with CCcompound1 were started on day 12 after tumor implantation and conducted for five days and then for another three days with two treatment-free days inserted between the two series of treatments. The results, shown in TABLE 4, indicate that between day 12 and 21 after tumor implantation, the prostate tumor caused about 5.7-g loss in body weight which corresponds to about 22% reduction. While administration of CCcompound1 in the amount of 1-mg/kg only partially prevented body weight loss, animals treated with 4-mg/kg of CCcompound1 actually gained 4.3-g. The results also show that 4-mg/kg of CCcompound1 reduced tumor weight about 48%. Overall the results indicate that in the PC-3 human prostate tumor model, CCcompound1 exerts potent anti-cachectic effects which are accompanied by significant anti-tumor effects.

TABLE 4
CCcompound1 prevents body weight loss in the PC-3 tumor model.

Days after
tumor
transplantation

| CCcompound1 mg/kg | Weight (g) | 12 | 21 |
|---|---|---|---|
| 0 | Total weight | 26.2 | 22.9 |
| | Tumor weight | 0.3 | 2.7 |
| | Body weight | 25.9 | 20.2 |
| 1.0 | Total weight | 26.3 | 25.3 |
| | Tumor weight | 0.4 | 2.2 |
| | Body weight | 25.9 | 23.1 |
| 2.0 | Total weight | 26.1 | 28.4 |
| | Tumor weight | 0.4 | 1.9 |
| | Body weight | 25.7 | 26.5 |
| 4.0 | Total weight | 26.9 | 32.4 |
| | Tumor weight | 0.2 | 1.4 |
| | Body weight | 26.7 | 31.0 |

### Example 5

### Effects of CCcompound1 on Body Weight as Well as Total and Tumor Weight in the T47D Human Estrogen Receptor Positive Breast Cancer Model

T47D human tumors were developed in mice as described above. Treatments with CCcompound1 were started on day 17 after tumor implantation and conducted for five days and then followed by an additional five days with 2 treatment-free days inserted between the 2 series of treatments. The results, shown in TABLE 5, indicate that between day 17 and 28 after tumor implantation, this breast tumor caused about a 2.5-g loss in body weight. Administration of 1-mg/kg of CCcompound1 already fully reversed body weight loss, while animals treated with 4-mg/kg of CCcompound1 gained 3.1-g. Treatment with 4-mg/kg of CCcompound1 also reduced tumor weight about 40%. Thus, in this human breast tumor model, CCcompound1 is a potent anti-cachectic agent and its effects on body weight are accompanied by significant anti-tumor effects.

TABLE 5
CCcompound1 prevents body weight loss in the T47D tumor model.

Days after
tumor
transplantation

| CCcompound1 mg/kg | Weight (g) | 17 | 28 |
|---|---|---|---|
| 0 | Total weight | 269 | 26.2 |
| | Tumor weight | 0.2 | 2.0 |
| | Body weight | 26.7 | 24.2 |
| 1.0 | Total weight | 26.5 | 29.3 |
| | Tumor weight | 0.2 | 1.6 |
| | Body weight | 26.3 | 27.7 |
| 2.0 | Total weight | 26.9 | 28.7 |
| | Tumor weight | 0.2 | 1.5 |
| | Body weight | 26.7 | 27.2 |
| 4.0 | Total weight | 27.0 | 30.9 |
| | Tumor weight | 0.2 | 1.2 |
| | Body weight | 26.8 | 29.7 |

1. A pharmaceutically acceptable composition to reduce or reverse cachexia or disease-induced body weight loss comprising a therapeutically effective amount of a thioxanthone compound containing a choline or choline analogue moiety.

2. The composition of claim 1, wherein the thioxanthone compound is [3-(3,4-dimethyl-9-oxo-9H-thioxanthen-2-yloxy)-2-hydroxypropyl]trimethyl-ammonium chloride or CCcompound1.

3. The composition of claim 1, wherein a dose will contain about 20-1000-mg of the thioxanthone compound per m² body surface.

4. The composition of claim 1, wherein a dose will contain about 50-500-mg of the thioxanthone compound per m² body surface.

5. The composition of claim 1, wherein the composition is administered at regular intervals as part of a more complex therapy to treat diseases such as cancer, AIDS, congestive heart failure, chronic obstructive pulmonary disease, cystic fibrosis, Crohn's disease, infections, rheumatoid arthritis, and tuberculosis.

6. The composition of claim 1, wherein the composition is administered to a subject during resting periods between a therapy cycle.

7. A method for reducing or reversing cachexia or disease-induced body weight loss comprising:
administering to a subject a pharmaceutically acceptable composition comprising a therapeutically effective amount of a thioxanthone compound containing a choline or choline analogue moiety.

8. The method of claim 7, wherein the thioxanthone compound is [3-(3,4-dimethyl-9-oxo-9H-thioxanthen-2-yloxy)-2-hydroxypropyl]trimethyl-ammonium chloride or CCcompound1.

9. The method of claim 7, wherein the composition is administered orally or rectally in the form of a tablet, a shaped gel-like composition or a liquid.

10. The method of claim 7, wherein the composition is injected via intravenous, intraarterial, intraperitoneal, subcutaneous, intramuscular, or intradermal routes.

11. The method of claim 10, wherein the composition is administered by infusion or an implanted minipump in a controlled fashion.

12. The method of claim 7, wherein the composition is administered in a dose containing about 20-1000-mg of the thioxanthone compound per m² body surface.

13. The method of claim 7, wherein the composition is administered in a dose containing about 50-500-mg of the thioxanthone per m² body surface.

14. The method of claim 7, wherein the composition is administered at regular intervals as part of a more complex therapy to treat cancer, AIDS, congestive heart failure, chronic obstructive pulmonary disease, cystic fibrosis, Crohn's disease, infections, rheumatoid arthritis, or tuberculosis.

15. The method of claim 7, wherein the composition is administered to the subject during resting periods between a therapy cycle.

16. The method of claim 7, wherein the composition is administered to an elderly person experiencing cachexia without disease or starvation.

17. The method of claim 7, wherein the composition is administered to the subject with cancer.
